(19) United States
(12) Patent Application Publication
(10) Pub. No.: US 2023/0272339 A1
(43) Pub. Date: Aug. 31, 2023

(54) COMPOSITIONS AND METHODS FOR CELLULAR COMPONENT TRANSFER THERAPY

(71) Applicant: The Johns Hopkins University, Baltimore, MD (US)

(72) Inventors: Mandeep SINGH, Balitmore (MD); Seth Blackshaw, Baltimore, MD (US); Robert Johnston, Lutherville, MD (US); Jiang Qian, Lutherville, MD (US); Ying Liu, Baltimore, MD (US)

(73) Assignee: THE JOHNS HOPKINS UNIVERSITY, Baltimore, MD (US)

(21) Appl. No.: 18/310,048

(22) Filed: May 1, 2023

Related U.S. Application Data
(63) Continuation of application No. PCT/US21/57586, filed on Nov. 1, 2021.
(60) Provisional application No. 63/108,415, filed on Nov. 1, 2020.

(51) Int. Cl. C12N 5/0793

(52) U.S. Cl. CPC C12N 5/062; C12N 2513/00; C12N 2509/00

(57) ABSTRACT

The present disclosure provides methods for generating retinal cell clusters for use in cellular component transfer therapy, retinal cell clusters generated by such methods, and compositions comprising such retinal cell clusters. The present disclosure also provides uses of the retinal cell clusters and compositions comprising thereof for preventing and/or treating inherited retinal degenerative diseases.

### CROSS-REFERENCE TO RELATED APPLICATIONS

This application is a Continuation of International Patent Application No. PCT/US2021/057586, filed on Nov. 1, 2021, which claims priority to U.S. Provisional Patent Application No. 63/108,415, filed Nov. 1, 2020, the contents of each of which is are incorporated by reference in its their entirety entireties, and to each of which priority is claimed.

### INTRODUCTION

The present disclosure provides methods for generating retinal cells for use in cellular component transfer therapy, retinal cells generated by such methods, and compositions comprising such retinal cells. The present disclosure also provides uses of the retinal cells and compositions comprising thereof for preventing and/or treating inherited or acquired retinal degenerative diseases.

### BACKGROUND

Photoreceptor cell transplantation is currently being developed as a treatment for blindness resulting from a variety of inherited or acquired retinal degenerative diseases. In one approach, subretinal transplantation of retinal cells results in the therapeutic transfer of cytoplasm from donor to host cells. In contrast to any expectation that donor cells will lodge as independently functionally photoreceptors, cellular component transfer therapy (“CCTT”) acts by repairing the dysfunctional photoreceptor cells already present in the recipient's retina. Despite recent advances in cell culture strategies allowing for the production retinal organoids as a source of donor photoreceptor cells, there remains a need for improved methods for generating retinal cells suitable for efficiently and effectively treating inherited or acquired retinal degenerative diseases via CCTT.

### SUMMARY OF THE INVENTION

The present disclosure provides methods for generating retinal cells for use in CCTT, retinal cells generated by such methods, and compositions comprising such retinal cells. The present disclosure also provides uses of the retinal cells and compositions comprising thereof for preventing and/or treating inherited or acquired retinal degenerative diseases.

In certain embodiments, the present disclosure is directed to an in vitro method to produce retinal cell populations wherein at least about 60% of the cells of the retinal cell populations express a marker of photoreceptor cell identity, comprising: (a) generating a three-dimensional retinal organoid; (b) dissociating the three-dimensional retinal organoid; and (c) selecting for a retinal cell population wherein at least about 60% of the cells of the retinal cells express a marker of photoreceptor cell identity.

In certain embodiments, the marker of photoreceptor cell identity is CRX or RCVRN.

In certain embodiments, the three-dimensional retinal organoid is enzymatically dissociated. In certain embodiments, the enzyme papain or trypsin. In certain embodiments, the retinal cells are contacted with a composition to ensure that the cells remain in a dissociated cell suspension. In certain embodiments, the composition to ensure the cells remain in a dissociated cell suspension comprises DNAse. In certain embodiments, the retinal cells are contacted with a composition to enhance the survival of the cells in a dissociated cell suspension. In certain embodiments, the composition to enhance the survival of the cells in a dissociated cell suspension comprises a B-27 cell culture supplement (Thermo Fisher Scientific) or an N-2 cell culture supplement (Thermo Fisher Scientific).

In certain embodiments, the three-dimensional retinal organoid reaches between about DD 45 and DD 300 prior to being dissociated. In certain embodiments, the three-dimensional retinal organoid reaches about DD 90 to about DD 140 prior to being dissociated.

In certain embodiments, the retinal cell population consists of at least about 70% single cells. In certain embodiments, the retinal cell population consists of at least about 80% single cells. In certain embodiments, the retinal cell population consists of at least about 90% single cells.

In certain embodiments, the retinal cell population comprises about 15% to about 45% cone photoreceptor cells. In certain embodiments, of the cone photoreceptor cells: (a) more than about 30% express CNGA3; (b) more than about 30% express CNGB3; (c) more than about 20% express ARR3; (d) at least about 3% express THRB; and/or (e) at least about one cell expressing S-opsin.

In certain embodiments, the retinal cell population comprises about 55% to about 85% rod photoreceptor cells. In certain embodiments, of the rod photoreceptor cells: (a) more than about 50% express NRL; (b) more than about 40% express NR2E3; (c) more than about 20% express PDE6B; (d) more than about 30% expression of CNGA1; and/or (e) at least about one cell expressing RHO.

In certain embodiments, the retinal cell population comprises: (a) less than about 10% of the cells express a marker of bipolar cell identity; (b) less than about 20% of the cells express a marker of Muller glia cell identity; (c) less than about 10% of the cells express a marker of retinal microglia cell identity; (d) less than about 5% of the cells express a marker of forebrain neural progenitor cell identity; (e) less than about 3% of the cells express a marker of retinal progenitor cell identity. In certain embodiments: the marker of bipolar cell identity is one or more of ISL1, SEBOX, CAPB5, BHLHE23, GRM6, SCGN, NRN1L, GRIK1, KLHDC8A, and PROX1; the marker of Muller glia cell identity is one or more of AQP4, PRDX6, VIM, HES1, SLC1A3, GLUL, CLU, RLBP1 and LHX2; the marker of retinal microglia cell identity is one or more of PTPRC, MPEG1, and CXCR1; the marker of forebrain neural progenitor cell identity is one or more of NKX2.2, RGCC, NEUROD1, BTG2, GADD45A, and GADD45G; and the marker of retinal progenitor cell identity is one or more of HOPX, CDK4, CCND2, VSX2, and CCND1.

In certain embodiments, the retinal cell populations described herein comprise: (a) less than about 10% of the cells express a marker of horizontal cell identity; (b) less than about 10% of the cells express a marker of ganglion cell identity; (c) less than about 5% of the cells express a marker of retinal amacrine cell identity: (d) less than about 5% of the cells express a marker of astrocyte cell identity; (e) less than about 5% of the cells express a marker of pericyte cell identity; (f) less than about 5% of the cells express a marker of vascular cell identity; and/or (g) less than about 10% of the cells express a marker of retinal pigment epithelium cell identity. In certain embodiments, the marker of horizontal cell identity is one or more of ONECUT2, ONECUT1, and LHX1; the marker of ganglion cell identity is one or more of POU4F1, THY1, BRN3B, and SNCG; the marker of retinal amacrine cell identity is one or more of TFAP2B, ELAVL3, and ELAVL4; the marker of retinal pigment epithelium cell identity is one or more of BEST1, TIMP3, GRAMD3, and PITPNA.

In certain embodiments, the retinal cell population comprises no more than about one cell expressing CD15 or CD133, and/or less than about 30% of cells expressing A2B5 and CD38.

In certain embodiments, the stem cells are selected from human, nonhuman primate or rodent nonembryonic stem cells; human, nonhuman primate or rodent embryonic stem cells; human, nonhuman primate or rodent induced pluripotent stem cells; and human, nonhuman primate or rodent recombinant pluripotent cells.

In certain embodiments, the present disclosure is directed to a cell population of in vitro differentiated retinal cells, wherein said in vitro differentiated retinal cells are obtained by a method described herein.

In certain embodiments, the present disclosure is directed to a composition comprising the in vitro differentiated retinal cells, wherein said in vitro differentiated retinal cells are obtained by a method described herein. In certain embodiments, the composition is a pharmaceutical composition further comprising a pharmaceutically acceptable carrier.

In certain embodiments, the present disclosure is directed to methods of preventing and/or treating an inherited or acquired retinal degenerative disease in a subject, comprising administering to the subject an effective amount of one of the following: (a) a retinal cell population as described herein; or (b) the composition of retinal cells as described herein. In certain embodiments, the inherited retinal degenerative disease is selected from retinitis pigmentosa, choroideremia, Stargardt disease, cone-rod dystrophy, and Leber Congenital Amaurosis. In certain embodiments, the acquired retinal degenerative disease is age-related macular degeneration.

### BRIEF DESCRIPTION OF THE DRAWINGS

FIG. 1 depicts an exemplary model of retinal cellular component transfer therapy.

FIGS. 2A-2B depict the implantation of donor photoreceptor cells harvested from NRL-GFP mice or Rho-GFP mice and transplanted into wild-type adult mice (FIG. 2A) and data indicating that cellular repair efficacy exceeds the predicted threshold for vision repair (FIG. 2b).

FIG. 3 illustrates the predicted and established efficacy of CCTT in multiple mutation classes, including mitochondrial mutations.

FIG. 4 depicts the results of a xenotransplantation experiment to validate that physiologically and/or therapeutically relevant proteins are susceptible to CCTT. A close analogue of proposed CCTT human donor cells was transplanted into recipient wild-type mice. The recipient retinae were extracted after 2-4 weeks and the grafts were removed. The recipient retinae were lysed and processed by bulk proteomics. Transferred cellular proteins include those with functions relating to membrane-bound organelles, endoplasmic reticulum, extracellular matrix, and other cellular compartments or components

FIG. 5 depicts the results of a homologous modeling experiment where Donor Postnatal Rho.GFP photoreceptor cells that were functionally competent were transplanted into GNAT1/GNAT2 double knockout adult recipient mice. RGC function was measured after 4-6 weeks in situ: Light responses from a RGC in a transplanted retina. Five consecutive recordings. Upper, cell responses; lower, light stimulation pattern. Holding potential is set at −70 mV, which is close to the reversal potential of Cl—, to record excitatory postsynaptic current (EPSC). RGCs were recorded in Ames' buffer at 32-35° C. Photopic full-field white light stimulations (2 second duration, 2 second interval) were used to trigger responses.

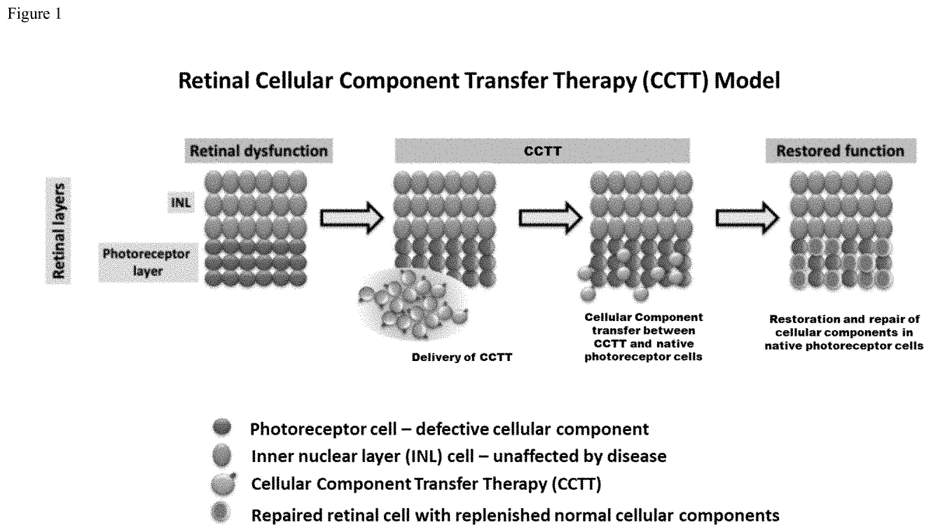

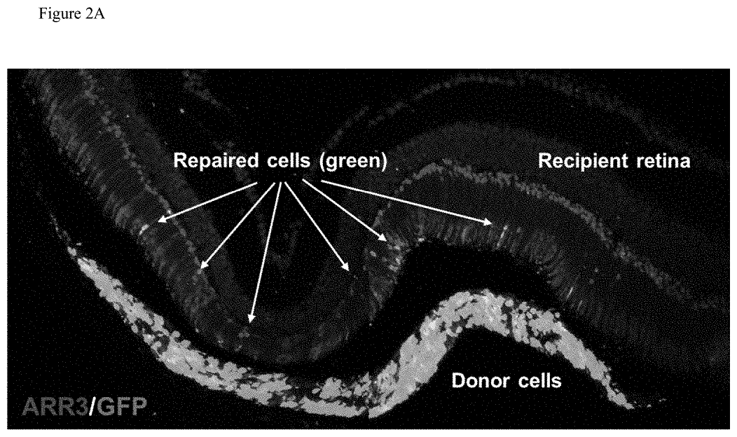

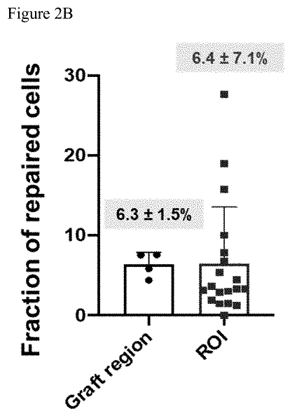

### DETAILED DESCRIPTION

The present disclosure provides methods for generating retinal cells for use in cellular component transfer therapy, retinal cells generated by such methods, and compositions comprising such retinal cells. The present disclosure also provides uses of the retinal cells and compositions comprising thereof for preventing and/or treating inherited or acquired retinal degenerative diseases.

Non-limiting embodiments of the presently disclosed subject matter are described by the present specification and Examples. For purposes of clarity of disclosure and not by way of limitation, the detailed description is divided into the following subsections:
1. Definitions;
2. Methods of Generating Retinal Cells;
3. Retinal Cell Populations & Retinal Cell Compositions; and
4. Methods of Treating Inherited Retinal Degenerative Diseases.

### 1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the compositions and methods of the present disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of photoreceptor cells, or a population of undifferentiated stem cells. Alternatively, the population may comprise more than one cell type, for example a mixed cell population. In certain embodiments, the cells in the population of cells are entirely dissociated from each other, e.g., the population of cells is a suspension of individual cells. In certain embodiments, the population of cells comprises undissociated clusters of cells. For example, but not by way of limitation, such populations of cells can comprise up to about 1%, up to about 2%, up to about 3%, up to about 4%, up to about 5%, up to about 6%, up to about 7%, up to about 8%, up to about 9%, or up to about 10% of the cells in the population present as undissociated clusters comprising up to about 10 cells. In certain embodiments, such populations of cells can comprise up to about 1%, up to about 2%, up to about 3%, up to about 4%, up to about 5%, up to about 6%, up to about 7%, up to about 8%, up to about 9%, or up to about 10% of cells in the population present as undissociated clusters comprising up to about 25 cells.

As used herein, the term “stem cell” refers to a cell with the ability to divide for indefinite periods in culture and to give rise to specialized cells.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human embryo. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell formed by the introduction of certain embryonic genes (such as but not limited to OCT4, SOX2, and KLF4 transgenes) (see, for example, Takahashi and Yamanaka Cell 126, 663-676 (2006), herein incorporated by reference) into a somatic cell.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation.

As used herein, the term “proliferation” refers to an increase in cell number.

As used herein, the term “undifferentiated” refers to a cell that has not yet developed into a specialized cell type.

As used herein, the term “differentiation” refers to a process whereby an unspecialized embryonic cell acquires the features of a specialized cell such as a retinal, heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as a retinal cell. In references to a stem cell, “directed differentiation” refers to the use of small molecules, growth factor proteins, and other growth conditions to promote the transition of a stem cell from the pluripotent state into a more mature or specialized cell fate.

As used herein, the term “inducing differentiation” in reference to a cell refers to changing the default cell type (gene expression profile and/or phenotype) to a non-default cell type (gene expression profile and/or phenotype). Thus, “inducing differentiation in a stem cell” refers to inducing the stem cell (e.g., human stem cell) to divide into progeny cells with characteristics that are different from the stem cell, such as in gene expression profile (e.g., change in gene expression as determined by genetic analysis such as a microarray) and/or phenotype (e.g., change in the number or presence of a protein marker, e.g., a cell surface marker, of rod or cone photoreceptor cells, such as CRX, RCVRN, CNGA3, CNGB3, ARR3, THRB, S-opsin, NRL, NR2E3, PDE6B, CNGA1, and RHO).

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, a spinner flask, and the like, and contains nutrients to nourish and support the cells. Culture medium may also include growth factors added to produce desired changes in the cells.

As used herein, the term “contacting” a cell or cells with a compound (e.g., at least one inhibitor, activator, and/or inducer) refers to providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, retina formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) an ultimate parent cell in a cell line, tissue (such as a dissociated embryo, or fluids using any manipulation, such as, without limitation, single cell isolation, culture in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease, a treatment can prevent deterioration due to a disease in an affected or diagnosed subject or a subject suspected of having the disease, but also a treatment may prevent the onset of the disease or a symptom of the disease in a subject at risk for the disease or suspected of having the disease.

### 2. Methods of Generating Retinal Cells

2.1. Three-Dimensional Cell Culture of Retinal Cells

The present disclosure provides for in vitro methods for inducing differentiation of stem cells (e.g., human stem cells). The presently disclosed subject matter provides in vitro methods for inducing differentiation of stem cells to produce retinal cells, e.g., rod and/or cone photoreceptor cells. In certain embodiments, the stem cells are pluripotent stem cells. In certain embodiments, the pluripotent stem cells are selected from embryonic stem cells (ESCs), induced pluripotent stem cells (iPSCs), and combinations thereof. In certain embodiments, the stem cells are multipotent stem cells. Non-limiting examples of stem cells that can be used with the presently disclosed methods include human, nonhuman primate or rodent nonembryonic stem cells, embryonic stem cells, induced nonembryonic pluripotent cells and engineered pluripotent cells. In certain embodiments, the stem cells are human stem cells. Non-limiting examples of human stem cells include human pluripotent stem cell (hPSC) (including, but not limited to human embryonic stem cells (hESC) and human induced pluripotent stem cells (hiPSC)), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the stem cell is an embryonic stem cell (ESC). In certain embodiments, the stem cell is a human embryonic stem cell (hESC). In certain embodiments, the stem cell is an induced pluripotent stem cell (iPSC). In certain embodiments, the stem cell is a human induced pluripotent stem cell (hiPSC).

In certain embodiments, the in vitro methods for inducing differentiation of stem cells to produce retinal cells of the present disclosure comprise the use of factors that promote rod and cone photoreceptor fate specification and survival. In certain embodiments, the in vitro methods for inducing differentiation of stem cells to produce retinal cells of the present disclosure comprise the use of factors that that suppress fate specification and survival of retinal interneurons, e.g., bipolar cells and retinal ganglion cells. In certain embodiments, the in vitro methods for inducing differentiation of stem cells to produce retinal cells of the present disclosure comprise the use of factors that that suppress fate specification and survival of retinal glia, e.g., Muller glia. In certain embodiments, the in vitro methods for inducing differentiation of stem cells to produce retinal cells of the present disclosure comprise the use of factors that that: (a) promote rod and cone photoreceptor fate specification and survival; suppress fate specification and survival of retinal interneurons, e.g., bipolar cells and retinal ganglion cells; and/or (c) suppress fate specification and survival of retinal glia, e.g., Muller glia.

In certain embodiments, the present disclosure is directed to the generation of three-dimensional retinal organoids, e.g., three dimensional human retinal organoids. For example, but not by way of limitation, the strategies for generating three-dimensional human retinal organoids can be employed as described in Eldred et al., Science, 362:6411 (2018); Zhong et al., Nat Commun., 5:4047 (2014); Reichman et al., Stem Cells, 35:1176-88 (2017); Wahlin et al., Sci Rep., 7:766 (2017); Hallam et al., Stem Cells, 36:1535-51 (2018); Kaya et al., Mol. Vis., 25: 663-678 (2019); or Regent et al., Mol Vis., 26: 97-105 (2020), each of which is incorporated herein by reference in its entirety. In certain embodiments, human retinal organoids are differentiated to achieve specific ratios of cone subtypes (red/Long, green/Medium, and blue/Short). For example, but not by way of limitation, culturing the organoid in the presence of low retinoic acid (RA), e.g., less than about 1 μM RA, leads to organoids having high red cones. In certain exemplary embodiments, culturing the organoid in high RA, e.g., greater than about 1 μM to about 20 μM RA, (or Knockout of CYP26a1) leads to organoids with high blue and green cones. In certain exemplary embodiments, culturing the organoid in RA through day 80 leads to a peripheral mix of red, green, and blue cones. In certain exemplary embodiments, culturing the organoid in high thyroid hormone (T3), e.g., greater than about 1 nM to about 1 μM T3, with high RA e.g., greater than about 1 μM to about 20 μM RA, leads to organoids with high green cones. In certain exemplary embodiments, culturing the organoid in high T3, e.g., greater than about 1 nM to about 1 μM T3, with low RA, e.g., less than about 1 μM RA, leads to organoids with high red cones. In certain exemplary embodiments, knock out of thyroid hormone receptor in the organoid leads to high blue cones.

In certain embodiments, the differentiation of stem cells to retinal organoids includes in vitro differentiation of stem cells to cells expressing at least one retinal organoid marker. In certain embodiments, the differentiation of stem cells to retinal organoids includes in vitro differentiation of stem cells to cells exhibiting at least one morphological characteristic associated with retinal organoid differentiation. In certain embodiments, the differentiation of stem cells to retinal organoids includes in vitro differentiation of stem cells to cells expressing at least one retinal organoid marker and exhibiting at least one morphological characteristic associated with retinal organoid differentiation. Non-limiting examples of retinal organoid markers include Nrl, Rho, Arr3, and combinations thereof. Non-limiting examples of retinal organoid morphological characteristics include: (a) the development of a multilayered retinal organoid anatomy comprising, e.g., a photoreceptor outer nuclear layer and nascent outer segments; and (b) retinal pigment epithelium (RPE) pigmentation development.

In certain embodiments, the stem cells are allowed to differentiate to attain a target differentiation stage of the cells of the retinal organoid of at least about 45 days to about 300 days. In certain embodiments, the stem cells are allowed to differentiate to attain a target differentiation stage of the cells of the retinal organoid of at least about 50 days to about 300 days. In certain embodiments, the stem cells are allowed to differentiate to attain a target differentiation stage of the cells of the retinal organoid of at least about 55 days to about 300 days. In certain embodiments, the stem cells are allowed to differentiate to attain a target differentiation stage of the cells of the retinal organoid of at least about 60 days to about 300 days. In certain embodiments, the stem cells are allowed to differentiate to attain a target differentiation stage of the cells of the retinal organoid of at least about 70 days to about 300 days. In certain embodiments, the stem cells are allowed to differentiate to attain a target differentiation stage of the cells of the retinal organoid of at least about 75 days to about 300 days. In certain embodiments, the stem cells are allowed to differentiate to attain a target differentiation stage of the cells of the retinal organoid of at least about 80 days to 300 days. In certain embodiments, the stem cells are allowed to differentiate to attain a target differentiation stage of the cells of the retinal organoid of at least about 85 days to about 300 days. In certain embodiment, the stem cells are allowed to differentiate to attain a target differentiation stage of the cells of the retinal organoid of at least about 90 days, at least about 91 days, at least about 93 days, at least about 94 days, at least about 95 days, at least about 96 days, at least about 97 days, at least about 98 days, at least about 99 days, at least about 100 days, at least about 101 days, at least about 102 days, at least about 103 days, at least about 104 days, at least about 105 days, at least about 106 days, at least about 107 days, at least about 108 days, at least about 109 days, at least about 110 days, at least about 111 days, at least about 112 days, at least about 113 days, at least about 114 days, at least about 115 days, at least about 116 days, at least about 117 days, at least about 118 days, at least about 119 days, at least about 120 days, at least about 121 days, at least about 122 days, at least about 123 days, at least about 124 days, at least about 125 days, at least about 126 days, at least about 128 days, at least about 129 days, at least about 130 days, at least about 131 days, at least about 132 days, at least about 133 days, at least about 134 days, at least about 135 days, at least about 136 days, at least about 137 days, at least about 138 days, at least about 139 days, or at least about 140 days. The duration of differentiation can be noted as “DD”, e.g., allowed cells to differentiate to attain a target differentiation stage of the cells of the retinal organoid of at least about 50 days (“DD50”) to about 300 days (“DD300”).

2.2. Dissociation of Retinal Organoids

In certain embodiments, the present disclosure is directed to the generation of populations of retinal cells via the dissociation of the above-described retinal organoids. In certain embodiments, such dissociation involves the disruption of the laminar organization of cells in the organoid. In certain embodiments, such retinal organoids are dissociated by the addition of specific enzymes and/or additives that ensure that the cells remain in dissociated cell suspension rather than as aggregates. For example, but not by way of limitation, enzymes useful in connection with the dissociation of retinal organoids include papain and trypsin. Compositions useful in ensuring that the cells remain in a dissociated cell suspension include compositions comprising DNAse. Compositions useful to enhance the survival of the cells in a dissociated cell suspension include compositions comprising a B-27 cell culture supplement (Thermo Fisher Scientific) or an N-2 cell culture supplement (Thermo Fisher Scientific).

In certain embodiments, the populations of retinal cells resulting from dissociation of the retinal organoids of the present disclosure will contain at least 70% single cells, relative to the total number of cells (including doublet cells, triplet cells, and larger order undissociated clusters of cells). In certain embodiments, the cell populations of the present disclosure will contain between 70%-80% single cells, relative to the total number of cells (including doublet cells, triplet cells, and larger order undissociated clusters of cells). In certain embodiments, the cell populations of the present disclosure will contain between 70%-85% single cells, relative to the total number of cells (including doublet cells, triplet cells, and larger order undissociated clusters of cells). In certain embodiments, the cell populations of the present disclosure will contain between 70%-90% single cells, relative to the total number of cells (including doublet cells, triplet cells, and larger order undissociated clusters of cells). In certain embodiments, the cell populations of the present disclosure will contain between 70%-95% single cells, relative to the total number of cells (including doublet cells, triplet cells, and larger order undissociated clusters of cells). In certain embodiments, the cell populations of the present disclosure will contain between 70%-100% single cells, relative to the total number of cells (including doublet cells, triplet cells, and larger order undissociated clusters of cells).

In certain embodiments, the retinal cell populations resulting from dissociation of the retinal organoids of the present disclosure will contain at least 80% single cells, relative to the total number of cells (including doublet cells, triplet cells, and larger order undissociated clusters of cells). In certain embodiments, the cell populations of the present disclosure will contain between 80%-85% single cells, relative to the total number of cells (including doublet cells, triplet cells, and larger order undissociated clusters of cells). In certain embodiments, the cell populations of the present disclosure will contain between 80%-90% single cells, relative to the total number of cells (including doublet cells, triplet cells, and larger order undissociated clusters of cells). In certain embodiments, the cell populations of the present disclosure will contain between 80%-95% single cells, relative to the total number of cells (including doublet cells, triplet cells, and larger order undissociated clusters of cells). In certain embodiments, the cell populations of the present disclosure will contain between 80%-100% single cells, relative to the total number of cells (including doublet cells, triplet cells, and larger order undissociated clusters of cells).

In certain embodiments, the retinal cell populations resulting from dissociation of the retinal organoids of the present disclosure will contain at least 85% single cells, relative to the total number of cells (including doublet cells, triplet cells, and larger order undissociated clusters of cells). In certain embodiments, the cell clus populations ters of the present disclosure will contain between 85%-90% single cells, relative to the total number of cells (including doublet cells, triplet cells, and larger order undissociated clusters of cells). In certain embodiments, the cell cl populations usters of the present disclosure will contain between 85%-95% single cells, relative to the total number of cells (including doublet cells, triplet cells, and larger order undissociated clusters of cells). In certain embodiments, the cell populations of the present disclosure will contain between 85%-100% single cells, relative to the total number of cells (including doublet cells, triplet cells, and larger order undissociated clusters of cells).

In certain embodiments, the retinal cell populations resulting from dissociation of the retinal organoids of the present disclosure will contain at least 90% single cells, relative to the total number of cells (including doublet cells, triplet cells, and larger order undissociated clusters of cells). In certain embodiments, the cell populations of the present disclosure will contain between 90%-95% single cells, relative to the total number of cells (including doublet cells, triplet cells, and larger order undissociated clusters of cells). In certain embodiments, the cell populations of the present disclosure will contain between 90%-100% single cells, relative to the total number of cells (including doublet cells, triplet cells, and larger order undissociated clusters of cells).

In certain embodiments, the retinal cell populations resulting from dissociation of the retinal organoids of the present disclosure will contain at least 95% single cells, relative to the total number of cells (including doublet cells, triplet cells, and larger order undissociated clusters of cells). In certain embodiments, the cell populations of the present disclosure will contain between 95%-100% single cells, relative to the total number of cells (including doublet cells, triplet cells, and larger order undissociated clusters of cells).

2.3 Selective Enrichment and/or Negative Selection of Certain Cell Types

In certain embodiments, the present disclosure is directed to the generation of retinal cell populations comprising specific cell types. For example, but not by way of limitation, such retinal cell populations can be selectively enriched for or negatively selected for specific cell types. In certain embodiments, the retinal cell populations are sorted, e.g., via fluorescence-activated cell sorting, to selectively enrich for and/or negatively select for specific cell types.

In certain embodiments, at least about 60% of the cells of the retinal cell populations of the present disclosure express a marker of photoreceptor cell identity. For example, but not by way of limitation, the marker of photoreceptor cell identity is CRX or RCVRN. In certain embodiments, at least about 65% of the cells of the retinal cell populations of the present disclosure express a marker of photoreceptor cell identity. In certain embodiments, at least about 70% of the cells of the retinal cell populations of the present disclosure express a marker of photoreceptor cell identity. In certain embodiments, at least about 75% of the cells of the retinal cell populations of the present disclosure express a marker of photoreceptor cell identity. In certain embodiments, at least about 80% of the cells of the retinal cell populations of the present disclosure express a marker of photoreceptor cell identity. In certain embodiments, at least about 85% of the cells of the retinal cell populations of the present disclosure express a marker of photoreceptor cell identity. In certain embodiments, at least about 90% of the cells of the retinal cell populations of the present disclosure express a marker of photoreceptor cell identity. In certain embodiments, at least about 90% of the cells of the retinal cell populations of the present disclosure express a marker of photoreceptor cell identity. In certain embodiments, at least about 95% of the cells of the retinal cell populations of the present disclosure express a marker of photoreceptor cell identity. In certain embodiments, up to about 100% of the cells of the retinal cell populations of the present disclosure express a marker of photoreceptor cell identity.

In certain embodiments, at least about 15% to about 45% of the cells of the retinal cell populations of the present disclosure express at least one marker of cone photoreceptor cell identity. For example, but not by way of limitation, the marker of cone photoreceptor cell identity can be CNGA3, CNGB3, ARR3, THRB, or S-opsin. In certain embodiments, at least about 20% to about 45% of the cells of the retinal cell populations of the present disclosure express a marker of cone photoreceptor cell identity. In certain embodiments, at least about 25% to about 45% of the cells of the retinal cell populations of the present disclosure express a marker of photoreceptor cell identity. In certain embodiments, at least about 30% to about 45% of the cells of the retinal cell populations of the present disclosure express a marker of cone photoreceptor cell identity. In certain embodiments, at least about 35% to about 45% of the cells of the retinal cell populations of the present disclosure express a marker of cone photoreceptor cell identity. In certain embodiments, at least about 40% to about 45% of the cells of the retinal cell populations of the present disclosure express a marker of cone photoreceptor cell identity.

In certain embodiments, at least about 30% of the cells of the retinal cell populations expressing at least one marker of cone photoreceptor cell identity express CNGA3. In certain embodiments, at least about 30% of the cells of the retinal cell populations expressing at least one marker of cone photoreceptor cell identity express CNGB3. In certain embodiments, at least about 20% of the cells of the retinal cell populations expressing at least one marker of cone photoreceptor cell identity express ARR3. In certain embodiments, at least about 3% of the cells of the retinal cell populations expressing at least one marker of cone photoreceptor cell identity express THRB. In certain embodiments, at least one cell of the retinal cell populations expressing at least one marker of cone photoreceptor cell identity expresses S-opsin.

In certain embodiments, at least about 30% of the cells of the retinal cell populations expressing at least one marker of cone photoreceptor cell identity express CNGA3, at least about 30% of the cells of the retinal cell populations expressing at least one marker of cone photoreceptor cell identity express CNGB3, at least about 20% of the cells of the retinal cell populations expressing at least one marker of cone photoreceptor cell identity express ARR3, at least about 3% of the cells of the retinal cell populations expressing at least one marker of cone photoreceptor cell identity express THRB, and at least one cell of the retinal cell populations expressing at least one marker of cone photoreceptor cell identity expresses S-opsin.

In certain embodiments, at least about 55% to about 85% of the cells of the retinal cell populations of the present disclosure express at least one marker of rod photoreceptor cell identity. For example, but not by way of limitation, the marker of rod photoreceptor cell identity can be NRL, NR2E3, PDE6B, CNGA1, or RHO. In certain embodiments, at least about 60% to about 85% of the cells of the retinal cell populations of the present disclosure express a marker of rod photoreceptor cell identity. In certain embodiments, at least about 65% to about 85% of the cells of the retinal cell populations of the present disclosure express a marker of rod photoreceptor cell identity. In certain embodiments, at least about 70% to about 85% of the cells of the retinal cell populations of the present disclosure express a marker of rod photoreceptor cell identity. In certain embodiments, at least about 75% to about 85% of the cells of the retinal cell populations of the present disclosure express a marker of rod photoreceptor cell identity. In certain embodiments, at least about 80% to about 85% of the cells of the retinal cell populations of the present disclosure express a marker of rod photoreceptor cell identity

In certain embodiments, at least about 50% of the cells of the retinal cell populations expressing at least one marker of rod photoreceptor cell identity express NRL. In certain embodiments, at least about 40% of the cells of the retinal cell populations expressing at least one marker of rod photoreceptor cell identity express NR2E3. In certain embodiments, at least about 20% of the cells of the retinal cell populations expressing at least one marker of rod photoreceptor cell identity express PDE6B. In certain embodiments, at least about 30% of the cells of the retinal cell populations expressing at least one marker of rod photoreceptor cell identity express CNGA1. In certain embodiments, at least one cell of the retinal cell cluster expressing at least one marker of rod photoreceptor cell identity expresses RHO.

In certain embodiments, at least about 50% of the cells of the retinal cell populations expressing at least one marker of rod photoreceptor cell identity express NRL, at least about 40% of the cells of the retinal cell populations expressing at least one marker of rod photoreceptor cell identity express NR2E3, at least about 20% of the cells of the retinal cell populations expressing at least one marker of rod photoreceptor cell identity express PDE6B, at least about 30% of the cells of the retinal cell populations expressing at least one marker of rod photoreceptor cell identity express CNGA1, and at least one cell of the retinal cell populations expressing at least one marker of rod photoreceptor cell identity expresses RHO.

In certain embodiments, the cells of the retinal cell populations of the present disclosure are selected such that they comprise no more than about 40% cells that express a marker of non-photoreceptor cell identity. For example, but not by way of limitation, markers of non-photoreceptor cell identity are those markers associated with: bipolar cells, Muller glia cells, retinal microglia, forebrain neural progenitor cells, retinal progenitor cells, horizontal cells, ganglion cells, retinal amacrine cells, and retinal pigment epithelium cells.

In certain embodiments, the cells of the retinal cell populations of the present disclosure are selected such that they comprise less than about 10% of bipolar cells. In certain embodiments, the marker associated with bipolar cell identity is one or more of ISL1, SEBOX, CAPB5, BHLHE23, GRM6, SCGN, NRN1L, GRIK1, KLHDC8A, and PROX.

In certain embodiments, the cells of the retinal cell populations of the present disclosure are selected such that they comprise less than about 20% Muller glia cells. In certain embodiments, the marker associated with Muller glia cell identity is one or more of AQP4, PRDX6, VIM, HES1, SLC1A3, GLUL, CLU, RLBP1 and LHX2.

In certain embodiments, the cells of the retinal cell populations of the present disclosure are selected such that they comprise less than about 10% retinal microglia cells.

In certain embodiments, the marker associated with retinal microglia cell identity is one or more of PTPRC, MPEG1, and CXCR1.

In certain embodiments, the cells of the retinal cell populations of the present disclosure are selected such that they comprise less than about 5% forebrain neural progenitor cells. In certain embodiments, the marker associated with forebrain neural progenitor cell identity is one or more of NKX2.2, RGCC, NEUROD1, BTG2, GADD45A, and GADD45G.

In certain embodiments, the cells of the retinal cell populations of the present disclosure are selected such that they comprise less than about 3% retinal progenitor cells. In certain embodiments, the marker associated with retinal progenitor cell identity is one or more of HOPX, CDK4, CCND2, VSX2, and CCND1.

In certain embodiments, the cells of the retinal cell populations of the present disclosure are selected such that they comprise less than about 10% horizontal cells. In certain embodiments, the marker associated with horizontal cell identity is one or more of ONECUT2, ONECUT1, and LHX1.

In certain embodiments, the cells of the retinal cell populations of the present disclosure are selected such that they comprise less than about 10% retinal ganglion cells. In certain embodiments, the marker associated with retinal ganglion cell identity is one or more of POU4F1, THY1, BRN3B, and SNCG.

In certain embodiments, the cells of the retinal cell populations of the present disclosure are selected such that they comprise less than about 5% retinal amacrine cells. In certain embodiments, the marker associated with retinal amacrine cell identity is one or more of TFAP2B, ELAVL3, and ELAVL4.

In certain embodiments, the cells of the retinal cell populations of the present disclosure are selected such that they comprise less than about 10% retinal pigment epithelium cells. In certain embodiments, the marker associated with retinal pigment epithelium cell identity is one or more of BEST1, TIMP3, GRAMD3, and PITPNA.

In certain embodiments, the cells of the retinal cell populations of the present disclosure are selected such that less than 30% of the cells express a marker associated with inflammatory cell identity. For example, but not by way of limitation, markers of inflammatory cell identity are: CD15, CD133, A2B5, and CD38. In certain embodiments, the cells of the retinal cell populations of the present disclosure are selected such that they comprise less than about 30% cells expressing A2B5 and/or CD38. In certain embodiments, the cells of the retinal cell populations of the present disclosure are selected such that they comprise no more than one cell expressing CD15 or CD133.

### 3. Retinal Cell Populations & Retinal Cell Compositions

The present disclosure provides a cell population of in vitro differentiated retinal cells, wherein at least about 50% (e.g., at least about 55%, at least about 60%, at least about 65%, at least about 70%, at least about 75%, at least about 80%, at least about 85%, at least about 90%, at least about 95%, or at least about 99%) of the differentiated cells express at least one marker of photoreceptor cell identity.

In certain embodiments, the present disclosure provides a cell population of in vitro differentiated retinal cells, wherein less than at least about 40% (e.g., less than about 35%, less than about 30%, less than about 25%, less than about 20%, less than about 15%, less than about 10%, less than about 5%, less than about 4%, less than about 3%, less than about 2%, less than about 1%, less than about 0.5%, or less than about 0.1%) of the differentiated cells express at least one marker of non-photoreceptor cell identity.

In certain embodiments, the population of in vitro differentiated retinal cells comprises from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ in vitro differentiated photoreceptor cells.

The presently disclosure also provides compositions comprising such populations of in vitro differentiated retinal cells. In certain embodiments, the population of in vitro differentiated retinal cells are obtained by the differentiation methods described herein. In certain embodiments, said composition is frozen. In certain embodiments, said composition further comprises at least one cryoprotectant, for example, but not limited to, dimethylsulfoxide (DMSO), glycerol, polyethylene glycol, sucrose, trehalose, dextrose, or a combination thereof.

In certain embodiments, the composition is a pharmaceutical composition that comprises a pharmaceutically acceptable carrier. The compositions can be used for preventing and/or treating an inherited or acquired retinal degenerative disease, e.g., retinitis pigmentosa, choroideremia, Stargardt disease, cone-rod dystrophy, Leber Congenital Amaurosis and age related macular degeneration, including, but not limited to “dry” age related macular degeneration and “wet” age related macular degeneration.

### 4. Method of Treating Inherited Retinal Degenerative Diseases

The cell populations and compositions disclosed herein can be used for preventing and/or treating inherited and/or acquired retinal degenerative diseases. For example, but not by way of limitation, the cell populations and compositions disclosed herein can be used for CCTT, which, without being bound by theory, is understood to act by repairing the dysfunctional photoreceptor cells present in a recipient's retina. Again, without being bound by theory, it is understood that the cell populations and compositions disclosed herein exert their therapeutic effect, at least in part, by transferring healthy cellular components, e.g., organelles including mitochondria along with other nuclear, cell membrane-bound, and/or cytoplasmic components, e.g., therapeutic proteins. Thus, the presently disclosed subject matter provides for methods of preventing and/or treating inherited and/or acquired retinal degenerative diseases. In certain embodiments, the methods comprise administering the presently disclosed stem-cell-derived retinal cells or compositions comprising thereof to a subject suffering from an inherited or acquired retinal degenerative disease. In certain embodiments, the composition is a pharmaceutical composition further comprising a pharmaceutically acceptable carrier.

CCTT is effective in multiple mutation classes. For example, CCT is effective in X-linked mutations, autosomal dominant (AD) mutations, autosomal recessive (AR) mutations, and non-mendelian, e.g., mitochondrial, mutations. In addition, with respect to AD mutations, CCTT is effective in haploinsufficiency or dominant negative mutations (e.g., dominant negative interference mutations and dominant negative toxicity mutations). CCTT has also been shown effective in transferring multiple types of cellular components, e.g., membrane-bound proteins, nuclear-localized proteins, cytoplasmic proteins. CCTT is also effective in transferring cellular components to both types of photoreceptor cells, i.e., both rods and cones.

Non-limiting examples of inherited retinal degenerative diseases include retinitis pigmentosa, choroideremia, Stargardt disease, cone-rod dystrophy, and Leber Congenital Amaurosis. Non-limiting examples of acquired retinal degenerative diseases include, age related macular degeneration, including, but not limited to “dry” age related macular degeneration and “wet” age related macular degeneration.

The populations of retinal cells or compositions described herein can be administered in any physiologically acceptable vehicle. The cells or compositions of the present disclosure can be administered via localized injection or via subretinal transplant. In certain embodiments, the populations of cells or compositions will be resuspended in media and transplanted into the subretinal space using a device that preserves their biologic activity and ensures on-target placement. In certain embodiments, the device will be comprised of biocompatible materials. In certain embodiments, the device will accomplish the transplant with limited shear stress on cells, e.g., it will comprise a low-friction passage. An exemplary device for subretinal transplant is described in International Patent Application No. PCT/US2019/045074 (Published as WO2020028892), which is incorporated herein by reference in its entirety.

The cells or compositions can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived retinal cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin.

Viscosity of the compositions, if desired, can be maintained at the selected level using a pharmaceutically acceptable thickening agent. Methylcellulose can be used because it is readily and economically available and is easy to work with. Other suitable thickening agents include, for example, hyaluronic acid, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the non-cellular derived components of the compositions should generally, but not exclusively, be selected to be chemically inert and thus not affect the viability or efficacy of the presently disclosed retinal cells. This will present no problem to those skilled in chemical and pharmaceutical principles, or problems can be readily avoided by reference to standard texts or by simple experiments (not involving undue experimentation), from this disclosure and the documents cited herein.

In certain embodiments, the composition comprises an effective amount of the retinal cells. As used herein, the term “effective amount” or “therapeutically effective amount” refers to an amount sufficient to affect a beneficial or desired clinical result upon treatment. An effective amount can be administered to a subject in at least one dose. In terms of treatment, an effective amount is an amount that is sufficient to palliate, ameliorate, stabilize, reverse or slow the progression of the inherited retinal degenerative disease, or otherwise reduce the pathological consequences of the inherited retinal degenerative disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the cells is an amount that is sufficient to improve the retinal function of a subject suffering from an inherited retinal degenerative disease. In certain embodiments, an effective amount of the cells is an amount that is sufficient to improve the retinal function of a subject suffering from an inherited or acquired retinal degenerative disease, e.g., the improved function can be about 1%, about 5%, about 10%, about 20%, about 30%, about 40%, about 50%, about 60%, about 70%, about 80%, about 90%, about 95%, about 98%, about 99% or about 100% of the retinal function of an individual not suffering from the inherited or acquired retinal degenerative disease.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the cells are administered to a subject. In certain embodiments, from about 1×10⁵ to about 1×10⁷ of the cells are administered to a subject suffering from an inherited or acquired retinal degenerative disease. In certain embodiments, from about 1×10⁶ to about 1×10⁷ of the cells are administered to a subject suffering from an inherited or acquired retinal degenerative disease. In certain embodiments, from about 1×10⁶ to about 4×10⁶ of the cells are administered to a subject suffering from an inherited or acquired retinal degenerative disease. The precise determination of what would be considered an effective dose may be based on factors individual to each subject, including their size, age, sex, weight, and condition of the particular subject. Dosages can be readily ascertained by those skilled in the art from this disclosure and the knowledge in the art.

### Examples

1. Cellular Repair Efficacy Exceeds the Predicted Threshold for Vision Repair

In order to estimate CCTT efficiency and validate the occurrence of CCTT between photoreceptor cells of the same species (i.e., homologous modeling), postnatal day 3 GFP-producing donor photoreceptor cells were harvested from NRL-GFP mice or Rho-GFP mice and transplanted into wild-type adult mice recipients. Histology was obtained at 2-3 weeks post transplantation, counting the number of recipient cells labeled with GFP through the process of CCTT. The number of recipient cells was counted per section and also per region of interest (ROI) to capture the inter-ROI variability in efficiency. Mouse cells were employed to avoid the vulnerability of donor human cells to severe immune rejection, which would compromise the aims of the instant experiment.

Homologous modeling of cellular component transfer shows average efficacy of about 6.3-6.4% and peak efficacy of almost 30% (the fraction of recipient photoreceptor cells that are repaired using this method of treatment). We predict that a repair efficiency level of 5% of recipient photoreceptor cells is the threshold for functional vision improvement.

2. CCTT Shows Mutation-Agnostic and Mutation Class-Agnostic Functional Efficacy in Both Classes of Recipient Photoreceptor Cells

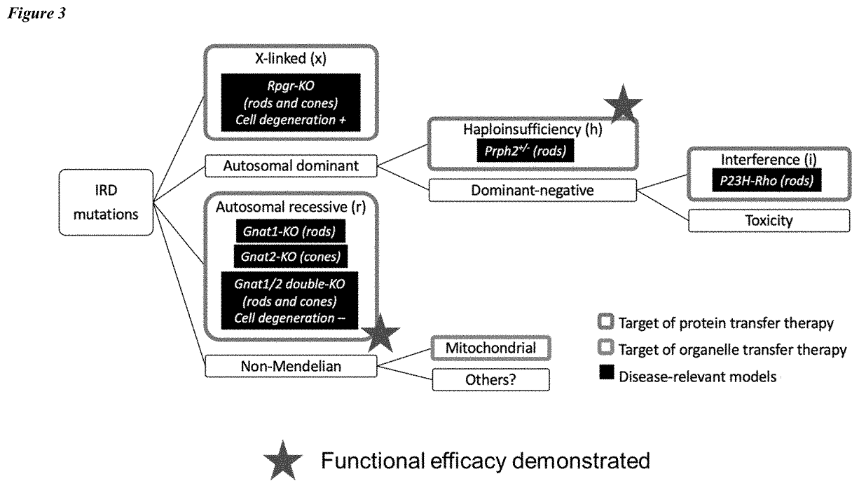

A series of experiments have been performed to validate the ubiquity of the treatment effect of CCTT. As described in Example 1, homologous modeling can be used to estimate the magnitude of functional effect following CCTT between photoreceptor cells of the same species. For example, CCTT efficacy in multiple mutation classes, e.g., autosomal dominant (AD) versus autosomal recessive (AR), has been validated using homologous modeling. Similarly, the occurrence of CCTT in multiple specific mutations, i.e., PRPH2 versus GNAT1 versus GNAT2 has also been validated using homologous modeling. CCTT efficacy in transferring multiple cellular components, e.g., membrane-bound protein (PRPH2) vs. nuclear-localized protein (GNAT1), has also been validated using homologous modeling. Finally, CCTT efficacy in both types of photoreceptor cells, i.e., both rods and cones, has been validated using homologous modeling. FIG. 3 illustrates the predicted efficacy of CCTT in multiple mutation classes, including mitochondrial mutations, based on the results described herein.

Postnatal day 3 GFP-producing donor photoreceptor cells were harvested from NRL-GFP mice or Rho-GFP mice and transplanted into wild-type adult mice recipients. Functional assays were obtained at 1-3 weeks post transplantation, by full-field electroretinography (FF-ERG), optokinetic nystagmus (OKN), and visual-guided behavior. Separate FF-ERG tests were used to dissect rod versus cone functional improvements. When possible, the same mice were tested at 1 week and 1-2 weeks thereafter, to promote rigor by validating reproducibility. Again, as noted in Example, 1 donor human cells were not employed in these experiments as they are vulnerable to severe immune rejection.

Functional testing in a mouse model of monogenic autosomal dominant IRD showed that the majority (>80%) of the treated mice regained vision and the mean visual function improved to −40% of wild-type vision level, reaching >85% of wild-type vision level in some mice. Functional testing in two mouse models of monogenic autosomal recessive IRD showed that the majority (>80%) of the treated mice regained vision and the mean visual function improved to −50% of wild-type vision level, reaching >95% of wild-type vision level in some mice. Evidence of functional repair occurs in both rod and cone photoreceptor cells of the recipient. In some mutations or mutations classes, functional repair may favor one class over the other. Moreover, by immunohistology and transcriptomics, evidence supporting the transfer of different cellular components was identified including intracellular cytoplasmic proteins (e.g. GFP); RNA, albeit at a low level; membrane-bound molecules (e.g. PRPH2); and nuclear-localized molecules including proteins (e.g. GNAT1).

3. Unbiased Proteomic Analysis Using a Cross-Species Approach Provides Evidence Supporting the Inter-Photoreceptor Transfer of Physiological Proteins

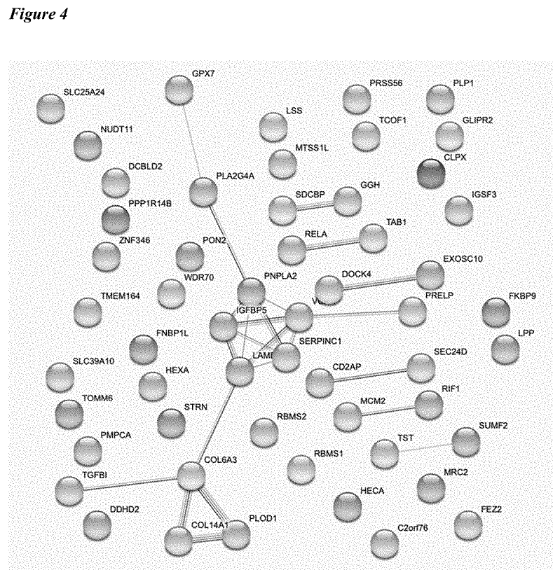

A xenotransplantation approach was employed in order to validate that physiologically and/or therapeutically relevant proteins, e.g., mammalian proteins, and not just (non-mammalian) labeling proteins such as GFP, are amenable to intercellular transfer by a mechanism consistent with CCTT. A close analogue of CCTT human donor cells described herein was transplanted into recipient wild-type mice. The recipient retinae were extracted after 2-4 weeks and the grafts were removed. The recipient retinae were lysed and processed by bulk proteomics. As illustrated in FIG. 4, this approach enabled detection and quantify the cellular components, e.g., proteins, of one species (human) that had been transferred into the cells of another species (mouse). Transferred cellular proteins include those with functions relating to membrane-bound organelles, endoplasmic reticulum, extracellular matrix, and other cellular compartments or components.

4. Whole-Cell Patch-Clamp Recordings from Transplanted Retinae Suggest that Repaired Cone Cells Respond to Light Stimulation

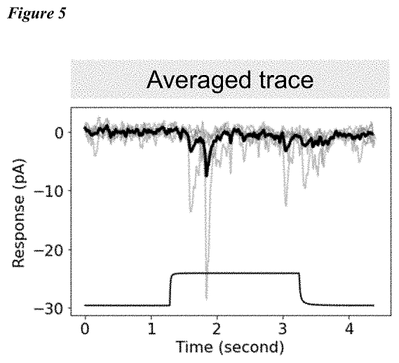

Homologous modeling was employed to validate that the downstream visual circuit is in fact reactivated following the repair of diseased recipient photoreceptor cells via a mechanism consistent with CCTT. Specifically, retinal ganglion cell (RGC) function was measured following cone repair by CCTT. RGC activity is indicative of visual circuit reactivation as RGCs would be the third order downstream neuron from any repaired cone. Donor Postnatal Rho.GFP photoreceptor cells that were functionally competent were transplanted into GNAT1/GNAT2 double knockout adult recipient mice. As illustrated in FIG. 5, RGC function was measured after 4-6 weeks in situ: Light responses from a RGC in a transplanted retina. Five consecutive recordings. Upper, cell responses; lower, light stimulation pattern. Holding potential is set at −70 mV, which is close to the reversal potential of Cl—, to record excitatory postsynaptic current (EPSC). RGCs were recorded in Ames' buffer at 32-35° C. Photopic full-field white light stimulations (2 second duration, 2 second interval) were used to trigger responses. The data depicted in FIG. 5 indicates that photoreceptor cells repaired by CCTT respond to light stimulation and that visually-evoked signals from those repaired cone cells are transmitted along the recipient visual circuit, thus re-establishing visual function in a previously inactive circuit.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. As one of ordinary skill in the art will readily appreciate from the present disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the present disclosures of which are incorporated herein by reference in their entireties for all purposes.

What is claimed is:

1. An in vitro method to produce retinal cell populations wherein at least about 60% of the cells of the retinal cell populations express a marker of photoreceptor cell identity, comprising:
a. generating a three-dimensional retinal organoid;
b. dissociating the three-dimensional retinal organoid; and
c. selecting for a retinal cell population wherein at least about 60% of the cells of the retinal cells express a marker of photoreceptor cell identity.

2. The method of claim 1, wherein the marker of photoreceptor cell identity is CRX or RCVRN.

3. The method of claim 1, wherein the three-dimensional retinal organoid is enzymatically dissociated.

4. The method of claim 3, wherein the enzyme is papain and/or trypsin.

5. The method of claim 3, wherein the retinal cells are contacted with a composition to ensure that the cells remain in a dissociated cell suspension.

6. The method of claim 5, wherein the composition is an enzyme.

7. The method of claim 6, wherein the enzyme is DNAse.

8. The method of claim 1, wherein the three-dimensional retinal organoid reaches between about DD 45 and DD 300 prior to being dissociated.

9. The method of claim 8, wherein the three-dimensional retinal organoid reaches about DD 90 to about DD 140 prior to being dissociated.

10. The method of claim 1, wherein the retinal cell population consists of at least about 70% single cells.

11. The method of claim 1, wherein the retinal cell population comprises about 15% to about 45% cone photoreceptor cells.

12. The method of claim 11, wherein, of the cone photoreceptor cells:
a. more than about 30% express CNGA3;
b. more than about 30% express CNGB3;
c. more than about 20% express ARR3;
d. at least about 3% express THRB; and/or
e. at least about one cell expressing S-opsin

13. The method of claim 1, wherein the retinal cell population comprises about 55% to about 85% rod photoreceptor cells.

14. The method of claim 1, wherein, of the rod photoreceptor cells:
a. more than about 50% express NRL;
b. more than about 40% express NR2E3;
c. more than about 20% express PDE6B;
d. more than about 30% expression of CNGA1; and/or
e. at least about one cell expressing RHO.

15. The method of claim 1, wherein the retinal cell population comprises:
a. less than about 10% of the cells express a marker of bipolar cell identity;
b. less than about 20% of the cells express a marker of Muller glia cell identity;
c. less than about 10% of the cells express a marker of retinal microglia cell identity;
d. less than about 5% of the cells express a marker of forebrain neural progenitor cell identity;
e. less than about 3% of the cells express a marker of retinal progenitor cell identity.

16. The method of claim 15, wherein:
a. the marker of bipolar cell identity is one or more of ISL1, SEBOX, CAPB5, BHLHE23, GRM6, SCGN, NRN1L, GRIK1, KLHDC8A, and PROX1;
b. the marker of Muller glia cell identity is one or more of AQP4, PRDX6, VIM, HES1, SLC1A3, GLUL, CLU, RLBP1 and LHX2;
c. the marker of retinal microglia cell identity is one or more of PTPRC, MPEG1, and CXCR1;
d. the marker of forebrain neural progenitor cell identity is one or more of NKX2.2, RGCC, NEUROD1, BTG2, GADD45A, and GADD45G; and/or
e. the marker of retinal progenitor cell identity is one or more of HOPX, CDK4, CCND2, VSX2, and CCND1.

17. The method of claim 1, wherein the retinal cell population comprises:
a. less than about 10% of the cells express a marker of horizontal cell identity;
b. less than about 10% of the cells express a marker of ganglion cell identity;
c. less than about 5% of the cells express a marker of retinal amacrine cell identity:
d. less than about 5% of the cells express a marker of astrocyte cell identity;
e. less than about 5% of the cells express a marker of pericyte cell identity;
f. less than about 5% of the cells express a marker of vascular cell identity; and/or
g. less than about 10% of the cells express a marker of retinal pigment epithelium cell identity.

18. The method of claim 17, wherein
a. the marker of horizontal cell identity is one or more of ONECUT2, ONECUT1, and LHX1;
b. the marker of ganglion cell identity is one or more of POU4F1, THY1, BRN3B, and SNCG;
c. the marker of retinal amacrine cell identity is one or more of TFAP2B, ELAVL3, and ELAVL4;
d. the marker of retinal pigment epithelium cell identity is one or more of BEST1, TIMP3, GRAMD3, and PITPNA.

19. The method of claim 1, wherein the retinal cell population comprises:
a. no more than about one cell expressing CD15 or CD133; and/or
b. less than about 30% of cells expressing A2B5 and CD38.

20. The method of claim 1, wherein the stem cells are selected from human, nonhuman primate or rodent nonembryonic stem cells; human, nonhuman primate or rodent embryonic stem cells; human, nonhuman primate or rodent induced pluripotent stem cells; embryonic stem cells, induced pluripotent stem cells; and human, nonhuman primate or rodent recombinant pluripotent cells.
